(19) United States
(12) Patent Application Publication
(10) Pub. No.: US 2023/0106284 A1
(43) Pub. Date: Apr. 6, 2023

(54) SYSTEM AND METHOD FOR GENERATING POTENTIAL DRUG COMPOSITIONS FOR DISEASE TARGET

(71) Applicant: Innoplexus AG, Eschborn (DE)

(72) Inventors: Om Sharma, Pimpri-Chinchwad (IN); Aryamen Singh, Bareilly (IN); Ashu Srivastav, Pune (IN)

(73) Assignee: Innoplexus AG, Eschborn (DE)

(21) Appl. No.: 18/063,793

(22) Filed: Dec. 9, 2022

Related U.S. Application Data
(63) Continuation-in-part of application No. 17/377,804, filed on Jul. 16, 2021.
(60) Provisional application No. 63/052,993, filed on Jul. 17, 2020.

(51) Int. Cl. G16H 70/40; G16B 20/00; G16B 15/30

(52) U.S. Cl. CPC G16H 70/40; G16B 20/00; G16B 15/30

(57) ABSTRACT

A system for generating drug compositions for a disease target, the system comprises a database arrangement and a processor, wherein the processor is configured to receive information comprising one or more drugs associated with the disease target, identify a plurality of parameters associated with the disease target, using the database arrangement, construct a matrix to identify at least one of direct and indirect synergies of each of the drug with the plurality of parameters and assign weights thereby to each of the parameters with respect to each of the drug, based on the identified at least one of direct and indirect synergies, calculate a total score of each of the drug and rank the plurality of drugs based on the calculated total score and sort thereby the plurality of drugs. The processor then determines the one or more potential drug compositions on the basis of the sorted plurality of drugs.

### CROSS REFERENCE TO RELATED APPLICATIONS

This application is a continuation in part application based on U.S. non-provisional patent application Ser. No. 17/377,804 titled “METHOD AND SYSTEM FOR EVALUATING POTENTIAL DRUG COMPOSITIONS FOR TARGET DISEASE” and filed on Jul. 16, 2021, which is incorporated herein by reference. The said non-provisional application is based upon a provisional patent application No. U.S. 63/052,993 as filed on Jul. 17, 2020, which is herein incorporated by reference in its entirety.

### TECHNICAL FIELD

The present disclosure relates generally to drug compositions; and more specifically, to method and systems for generating potential drug compositions for a disease target.

### BACKGROUND

Traditionally, developing a new drug for a disease is a process that takes a very long time and requires a lot of money for the development. Typically, association at a molecular level between a drug and a disease on which the drug exerts a pharmaceutical effect plays a critical role in the prediction of new drug indications. In order to decipher how drugs exert their effect on diseases at a molecular level, it is important to understand how a drug acts on targets related to a disease phenotype, how a gene module causes an abnormal phenotype, and how, in consequence, the targets and causative genes interact with each other. Moreover, the use of multiple drugs and/or treatment modalities in the treatment of individual patients is an increasingly commonplace occurrence. Consequently, the pace of new drug development, from drug discovery to drug production, has accelerated greatly, and single diseases are now treated with multiple drugs targeting different biochemical pathways or different aspects in the pathophysiology of a disease.

Notably, a great deal of information on drugs and trials is available on public sources, e.g., scientific publications, databases. However, the sheer volume of such data is overwhelming such that the data cannot be accessed and correlated in an efficient and effective manner. Moreover, compounding the problem is that the data are in disparate sources making it extremely hard to piece together in order to derive a fuller picture.

Currently, while there are a variety of databases available which cover clinical and experimental information, these databases do not adequately cover specialized information pertaining to pharmaceutical drugs and structural biology. Although some systems are geared towards specific target diseases treatments, these systems do not provide specific and specialized information regarding drug efficacy, potency, and other aspects related to drug administration. Additionally, to ensure the accuracy of the data, it is beneficial to be able to evaluate figures, graphs, tables and text within the results section of the documents. In some cases, content repositories may maintain millions of documents with no intelligent way to access complete content.

Therefore, in light of the foregoing discussion, there exists a need to overcome the aforementioned drawbacks associated with generating potential drug compositions for a disease target.

### SUMMARY

The present disclosure seeks to provide a method for generating potential drug for a disease target. The present disclosure also seeks to provide a system for generating potential drug compositions for a disease target. An aim of the present disclosure is to provide a solution that overcomes at least partially the problems encountered in prior art.

According to an aspect, embodiments of the present disclosure provide a system implemented in a computing device for generating potential drug compositions for a disease target, the system comprises:

a database arrangement; and

a processor communicably coupled via a data communication network to the database arrangement, wherein the processor is configured to:
receive information comprising one or more drugs associated with the disease target;
identify a plurality of parameters associated with the disease target, using the database arrangement, wherein the plurality of parameters comprises at least one of SoC drug targets, dys-regulated genes, pathways, molecular functions, biological process, experimental studies and adverse events;
construct a matrix to identify at least one of direct and indirect synergies of each of the drug with the plurality of parameters and assign weights thereby to each of the parameters with respect to each of the drug, based on the identified at least one of direct and indirect synergies;
calculate a total score of each of the drug for the plurality of the parameters based on the assigned weights to each of the parameter;
rank the plurality of drugs based on the calculated total score and sort the plurality of drugs thereby based on the ranked plurality of drugs; and
determine the one or more potential drug compositions on the basis of the sorted plurality of drugs.

Optionally, the processor is further configured to validate the at least one potential drug composition for the target disease based on biological evidence and differential expression analysis.

Optionally, wherein the received information also comprises the drug targets associated therewith each of the drug.

Optionally, each of the parameter from the plurality of parameters comprises a library of associated parameters.

Optionally, the processor is configured to identify at least one of the direct and indirect synergies of each of the drug with each of the parameter from the library of associated parameters.

Optionally, the processor is configured to identify at least one of the direct and indirect synergies of each of the drug with each of the parameter based on one or more ontologies.

Optionally, the one or more ontologies corresponds to at least a drug ontology, a protein ontology and a gene ontology.

Optionally, the processor is further configured to employ asset prioritization to filter the first set of potential drug compositions to determine at least one potential drug composition for the target disease.

Optionally, the asset prioritization to filter the one or more potential drug compositions to determine at least one potential drug composition comprises filtering based on:
potential drug compositions with no active clinical trials against the target disease;
inhibitory mechanisms of the potential drug compositions;
experimental support for the effectiveness against the target disease;
adverse events reported in public domain against the potential drug composition;
overall survival reports related to the target disease; and
binding affinity of the potential drug compositions.

Optionally, the processor is further configured to associate the one or more potential drug composition with a plurality of targets using the biological evidence and the differential expression analysis to validate the at least one potential drug composition.

Optionally, the processor is configured to evaluate one or more potential drug compositions to be used in combination with each other at a specific ratio.

In another aspect, embodiments of the present disclosure provide a method for generating potential drug compositions for a disease target, the method comprises:
receiving information comprising one or more drugs associated with the disease target;
identifying a plurality of parameters associated with the disease target, using the database arrangement, wherein the plurality of parameters comprises at least one of SoC drug targets, dys-regulated genes, pathways, molecular functions, biological process, experimental studies and adverse events;
constructing a matrix to identify at least one of direct and indirect synergies of each of the drug with the plurality of parameters and assigning weights thereby to each of the parameters with respect to each of the drug, based on the identified at least one of direct and indirect synergies;
calculating a total score of each of the drug for the plurality of the parameters based on the assigned weights to each of the parameter;
ranking the plurality of drugs based on the calculated total score and sorting the plurality of drugs thereby based on the ranked plurality of drugs; and
determining the one or more potential drug compositions on the basis of the sorted plurality of drugs.

Optionally, the method comprises validating the at least one potential drug composition for the target disease based on biological evidence and differential expression analysis.

Optionally, the received information also comprises the drug targets associated therewith each of the drug.

Optionally, each of the parameter from the plurality of parameters comprises a library of associated parameters.

Optionally, the method comprises identifying at least one of the direct and indirect synergies of each of the drug with each of the parameter from the library of associated parameters.

Optionally, the method comprises identifying at least one of the direct and indirect synergies of each of the drug with each of the parameter based on one or more ontologies, and wherein the one or more ontologies corresponds to at least a drug ontology, a protein ontology and a gene ontology.

Optionally, the method comprises employing asset prioritization to filter the first set of potential drug compositions to determine at least one potential drug composition for the target disease.

Optionally, the asset prioritization to filter the one or more potential drug compositions to determine at least one potential drug composition comprises filtering based on:
potential drug compositions with no active clinical trials against the target disease;
inhibitory mechanisms of the potential drug compositions;
experimental support for the effectiveness against the target disease;
adverse events reported in public domain against the potential drug composition;
overall survival reports related to the target disease; and
binding affinity of the potential drug compositions.

Optionally, the method comprises evaluating one or more potential drug compositions to be used in combination with each other at a specific ratio.

Embodiments of the present disclosure substantially eliminate or at least partially address the aforementioned problems in the prior art, and enable efficient evaluation of potential drug compositions for the target disease.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a block diagram of a system of determining potential drug compositions in accordance with an embodiment of the present disclosure;

FIG. 2 is a schematic illustration of the steps of method, in accordance with an embodiment of the present disclosure;

FIG. 3 is an illustration of an exemplary diagram showing betweenness centrality for marker prioritization, in accordance with an embodiment of the present disclosure;

FIG. 4A-4F are illustrations of exemplary graphs showing differential expressions of DFX with pancreatic cancer associated target proteins CYP3A4, UGT1A1, UGT1A3, UGT1A9, CYP2C8 and CYP1A2, in accordance with an embodiment of the present disclosure;

FIG. 5 is an illustration of a graph showing survival probability when compared with high and low expressions of target protein UGT1A1, in accordance with an embodiment of the present disclosure;

FIG. 6 is a block diagram showing evaluation of potential drug compositions DFX and a chemotherapy agent for pancreatic cancer, in accordance with an embodiment of the present disclosure; and

FIG. 7 is a schematic illustration of a block diagram showing efficient synergistic target identification, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

According to an aspect, embodiments of the present disclosure provide a system implemented in a computing device for generating potential drug compositions for a disease target, the system comprises:

a database arrangement; and

a processor communicably coupled via a data communication network to the database arrangement, wherein the processor is configured to:
receive information comprising one or more drugs associated with the disease target;
identify a plurality of parameters associated with the disease target, using the database arrangement, wherein the plurality of parameters comprises at least one of SoC drug targets, dys-regulated genes, pathways, molecular functions, biological process, experimental studies and adverse events;
construct a matrix to identify at least one of direct and indirect synergies of each of the drug with the plurality of parameters and assign weights thereby to each of the parameters with respect to each of the drug, based on the identified at least one of direct and indirect synergies;
calculate a total score of each of the drug for the plurality of the parameters based on the assigned weights to each of the parameter;
rank the plurality of drugs based on the calculated total score and sort the plurality of drugs thereby based on the ranked plurality of drugs; and
determine the one or more potential drug compositions on the basis of the sorted plurality of drugs.

In another aspect, the present disclosure provides a method for generating potential drug compositions for a disease target, the method comprises:
receiving information comprising one or more drugs associated with the disease target;
identifying a plurality of parameters associated with the disease target, using the database arrangement, wherein the plurality of parameters comprises at least one of SoC drug targets, dys-regulated genes, pathways, molecular functions, biological process, experimental studies and adverse events;
constructing a matrix to identify at least one of direct and indirect synergies of each of the drug with the plurality of parameters and assigning weights thereby to each of the parameters with respect to each of the drug, based on the identified at least one of direct and indirect synergies;
calculating a total score of each of the drug for the plurality of the parameters based on the assigned weights to each of the parameter;
ranking the plurality of drugs based on the calculated total score and sorting the plurality of drugs thereby based on the ranked plurality of drugs; and
determining the one or more potential drug compositions on the basis of the sorted plurality of drugs.

Pursuant to the embodiments of the present disclosure, the disclosed method and system and method identify the dual and poly-combination using a protein (targets)/drug feature matrix in the most efficient and effective way. The overall architecture includes Standard of care (SoC), pre-clinical, clinical, biological parameters and so forth, which improves the therapeutic effect or treatment condition The method described herein aims to evaluate potential drug compositions for a target disease in an efficient manner. The method described herein is able to handle sheer volume of such data, wherein the data includes drug compositions, target diseases, publications, targets, pathways and so forth. Furthermore, the data is made accessible and correlation of the data is performed in an efficient and effective manner. Additionally, the present disclosure adequately covers specialized information pertaining to the potential drug compositions and their structural biology. The present disclosure is able to provide specific and specialized information related to the target disease regarding drug efficacy, potency, and other aspects related to drug administration. Additionally, to ensure the accuracy of the data, the present disclosure is able to evaluate figures, graphs, tables and text within the results section of the publications. Moreover, the present disclosure performs intelligent parsing for information through millions of documents in order to collect content related to the target disease and the potential drug composition.

Throughout the present disclosure, the term “target disease” refers to a disease that is particularly considered for evaluating drug composition related thereto, in order to treat the disease. It should be understood that the method and system of the present disclosure is capable of evaluating and analyzing input for any number of target diseases.

Throughout the present disclosure, the term “potential drug compositions” refers to possible drugs that could be administered to treat or diagnose the target disease. Herein, potential drug composition is a chemical substance that is not known for its use against the target disease, but could be used to treat, cure, prevent or diagnose the target disease. Furthermore, potential drug compositions are formulated using pre-formulation studies, excipient compatibility studies, dissolution testing and other quality testing of various development batches. Additionally, potential drug composition will not affect the ability of the potential drug composition to bind with a pharmacological target, wherein pharmacological target refers to a biochemical entity to which the potential drug composition first binds in a participant's body to elicit its effect. Herein, the participant is a person taking part in a clinical trial. Moreover, the participant may be a patient suffering from the target disease of any gender or age, or a person willing to participate of any gender or age, wherein selection is performed based on an eligibility criterion.

Throughout the present disclosure, the term “processor” refers to a computational element that is operable to respond to and processes instructions that drive the system. Optionally, the processor includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processor” may refer to one or more individual processors, processing devices and various elements associated with a processing device that may be shared by other processing devices. Additionally, the one or more individual processors, processing devices and elements are arranged in various architectures for responding to and processing the instructions that drive the system.

Throughout the present disclosure, the term “data communication network” refers to individual networks or a collection thereof interconnected with each other and functioning as a single large network. Optionally, such a data communication network is implemented by way of a wired communication network, wireless communication network, or a combination thereof. It will be appreciated that a physical connection is established for implementing the wired communication network, whereas the wireless communication network is implemented using electromagnetic waves. Examples of such data communication networks include, but are not limited to, Local Area Networks (LANs), Wide Area Networks (WANs), Metropolitan Area Networks (MANs), Wireless LANs (WLANs), Wireless WANS (WWANs), Wireless MANS (WMANs), the Internet, second-generation (2G) telecommunication networks, third generation (3G) telecommunication networks, fourth-generation (4G) telecommunication networks, fifth-generation (5G) telecommunication networks and Worldwide Interoperability for Microwave Access (WiMAX) networks.

Throughout the present disclosure, the term “database arrangement” as used herein, relates to an organized body of digital information regardless of the manner in which the data or the organized body thereof is represented. It refers to a collection of data that allows easy access, management, and updating of the data stored. Optionally, the database arrangement may be hardware, software, firmware, and/or any combination thereof. For example, the organized body of digital information may be in a form of a table, a map, a grid, a packet, a datagram, a file, a document, a list or in any other form. Optionally, the data in the database arrangement is organized into rows, columns, and tables. Additionally, optionally, the data in the database arrangement is indexed (namely, labelled) for easy access thereto. Optionally, the database arrangement comprises a set of processes (namely instructions) to create the plurality of databases and update thereto, query data from external sources, and process operational instructions provided thereto.

Optionally, the database arrangement is accessed electronically for, for example storing data, accessing data, and updating data, using a computing device. More optionally, such a computing device employs a database management system (DBMS) for creating and managing the database arrangement. Furthermore, optionally, the database arrangement is an object-oriented database, SQL database, relational database, distributed database, non-SQL database, cloud database. The plurality of databases includes any data storage software and systems, such as, for example, a relational database like IBM DB2®, Google Cloud and Oracle 9®. Furthermore, the database arrangement also includes a software program for creating and managing one or more databases. Optionally, the database arrangement may be operable to support relational operations, regardless of whether it enforces strict adherence to a relational model, as understood by those of ordinary skill in the art. Additionally, the database arrangement is populated by the elastic search libraries, elastic search databases, at least one relevant data element, topic-based web content and the likes. Optionally, the database arrangement is populated by the operational data associated with the URIs, URLs, and/or URNs and their related information.

The present invention includes a system implemented in a computing device for generating potential drug compositions for a disease target, the system comprises a database arrangement and a processor communicably coupled via a data communication network to the database arrangement, wherein the processor is configured to receive information comprising one or more drug. Optionally, the received information comprises information relating to investigational clinical drugs, approved drugs, and generic drugs. Herein, investigational drugs are drugs that have been tested in a laboratory and approved by an administration of a government for testing in participants during clinical trials, whereas the administration of a government is specific by country. Furthermore, approved drugs are drugs validated for therapeutic use by the administration of a government. Additionally, generic drugs are copies of original drugs that have the same exact dosage, intended use, effects, side effects, method of administration, risks, safety and strength as the original drug. Optionally, the received information comprises information related to disease targets. Optionally, the received information comprises related to at least one of drugs and disease targets.

The processor is configured to identify a plurality of parameters associated with the disease target, using the database arrangement, wherein the plurality of parameters comprises at least one of SoC drug targets, dys-regulated genes, pathways, molecular functions, biological process, experimental studies and adverse events. Optionally, each of the parameter from the plurality of parameters comprises a library of associated parameters. In an example, the SoC drug target comprises a library of parameters such as, but not limited to, Bevacizunab, Cetuximab, Fluorourcil, Nivolumab and so forth. In another example, the parameter molecular function comprises a library of parameters such as, but not limited to, gene expression data, DNA sequence data, RNA sequence data, protein-protein interaction, pathway data, gene ontology and so forth.

The processor is further configured to construct a matrix to identify at least one of direct and indirect synergies of each of the drug with the plurality of parameters and assign weights thereby to each of the parameters with respect to each of the drug, based on the identified at least one of direct and indirect synergies. Optionally, the processor is configured to construct a matrix such that the one or more drugs are placed in at least a row, while the plurality of parameters are placed in at least a column. Optionally, the processor is configured to construct a matrix such that the one or more drug targets are placed in at least a row, while the plurality of parameters are placed in at least a column. More optionally, the processor is configured to construct a matrix such that the one or more drugs and the associated drug targets, are placed in at least a row, while the plurality of parameters are placed in at least a column. In an embodiment, the one or more drugs and/or the targets associated therewith, are placed in at least one of the column, while the plurality of parameters are placed in at least one of the row. It will be appreciated that each of the parameter from the plurality of parameters further comprise a library of parameters associated therewith. According to an embodiment, the processor is configured to identify at least one of the direct and indirect synergies of each of the drug with each of the parameter from the library of associated parameters. In an instance, the processor is configured to identify at least one of the direct and indirect synergies of each of the drug with each of the parameter based on one or more ontologies. In another instance, the processor is configured to identify at least one of the direct and indirect synergies of each of the drug with each of the above parameters from the plurality of parameters based on the identified one or more criteria from the one of more ontologies, wherein the one or more criteria may include, but not limited to, clinical trials, expression, variation/mutation, target class/family, drug class, clinical studies, sequence structure based similarity, targetability, drug resistance, BBB penetration and so forth.

Optionally, the processor is configured identify the interaction of one or more drugs with each of the parameters from the plurality of parameters in order to identify the synergies, i.e., direct and indirect synergies. Accordingly, the processor thereafter assigns weights to each of the parameter based on the identified interaction of the one or more drug with each of the parameters from the library of parameters from each of the parameter. In an instance, wherever there is a positive interaction between one or more drug and each parameter from the plurality of parameter, the processor assigns therewith a unity weight, i.e., 1, to each of the parameter with respect to each of the drug. In another instance, wherever there is no interaction between the one or more drug and the plurality of parameters, the processor assigns therewith a zero weight, i.e., 0, to each of the parameter with respect to each of the drug. Optionally, the processor is also configured to assign the weight between the value of unity and zero, in an instance where there is indirect interaction between each of the drug and each of the parameter from the plurality of parameters.

In an example, the processor analyzes the drugs D1, D2, D3, D4 and so forth, and the plurality of parameters comprises SoC drug targets, DE genes, pathway, molecular function and biological process. Each of the parameter (SoC drug targets, DE genes, pathway and so forth) may further comprise library of associated parameters, for example, SoC drug parameters may further comprises associated parameters such as P1, P2, P3 and so forth. Further, molecular function may further comprise associated parameters such as, for example, MF1, MF2, MF3 and so forth. The processor is configured to analyze and identify the interactions of drug D1, D2, D3 and so forth with the parameters such as P1, P2, P3, MF1, MF2, MF3 and so forth. Based on the identified interaction and synergies (either direct or indirect), the processor assigns weights to each of the parameter (P1, P2, P3, MF1, MF2 . . . ) with respect to each of the drug. The processor is further configured to calculate a total score of each of the drug for the plurality of the parameters based on the assigned weights to each of the parameter. Furthermore, the processor then ranks the plurality of drugs based on the calculated total score and sort the plurality of drugs thereby based on the ranked plurality of drugs. In an instance, when the calculated total score is 57, wherein the total score of 57 indicates that a particular drug (which has been taken as consideration to interact or identify the synergies with the plurality of parameters) has been identified synergistic (or having interacted in a positive manner) with the 57 parameters from the plurality of parameters. The figure may also rise for more than 57 parameters when the indirect synergies are also identified between the one or more drug and the plurality of parameters. After, calculating the total score and ranking and sorting the one or more drugs based on the assigned total score, the processor is further configured to determine the one or more potential drug compositions on the basis of the sorted plurality of drugs. Beneficially, the present invention is configured to overcome the problems associated with lack of sufficient data size, as the present invention receives only the information associated with the drug and/or the targets and also get rid of the redundant data. Also, since the present invention makes the scores and rankings apparent to the user, the present invention is configured to provide the potential drug composition in a user-friendly format. Additionally, the biological interpretation of machine-learning models is thereby improved.

Optionally, the processor is further configured to employ asset prioritization to filter the first set of potential drug compositions to determine at least one potential drug composition for the target disease. wherein the asset prioritization to filter the one or more potential drug compositions to determine at least one potential drug composition comprises filtering based on:
potential drug compositions with no active clinical trials against the target disease;
inhibitory mechanisms of the potential drug compositions;
experimental support for the effectiveness against the target disease;
adverse events reported in public domain against the potential drug composition;
overall survival reports related to the target disease; and
binding affinity of the potential drug compositions.

Optionally, the processor is further configured to associate the one or more potential drug composition with a plurality of targets using the biological evidence and the differential expression analysis to validate the at least one potential drug composition. It will be appreciated that the processor evaluates the one or more potential drug compositions to be used in combination with each other at a specific ratio.

In an embodiment, the processor further comprises a discovery engine to identify a first set of potential drug compositions for the target disease. Herein, the term “discovery engine” refers to a sub-processor that is able to analyze large volumes of raw data, filter out information and provide a useful output. Optionally, the discovery engine employs machine learning and artificial intelligence algorithms. Furthermore, the discovery engine may provide recommendations of potential drug compositions with respect to the target disease, organize the information and lead to the discovery of new leads for potential drug compositions. Furthermore, the discovery engine comprises omics related information, identifying network-based proximity between the potential drug compositions with the target disease, identifying new uses for existing drugs, such as for example using Remdesivir for treating SARS-CoV-2, wherein Remdesivir was originally developed to treat hepatitis C.

Optionally, the discovery engine is configured to analyze failed clinical assets of drugs for the target disease, wherein the discovery engine filters clinical trials which have failed due to non-drug related reasons. Herein, the non-drug related reason may be lack of funding for the drugs, inability to choose inclusion or exclusion criteria in an eligibility criterion appropriately, failing to enroll a sufficient number of participants in a clinical trial, highly variable additional costs associated with recruitment of a participant in the clinical trial, inadequate employment of quantitative measures, and so forth. In an example, ‘pancreatic cancer’ is the target disease for which the first set of potential drug compositions is to be determined. Herein, keywords are used in websites which contain repositories of publicly available clinical trials. Subsequently, the keyword corresponding to pancreatic cancer is ‘Pancreatic cancer’, which is thereafter given as input to the website. Thereafter, approximately 2,482 pancreatic cancer related clinical trials may be identified and filtered. Subsequently, further filtering of clinical trials is performed based on a participant receiving several drugs at a time, which is denoted by a trial status ‘Interventional’. Herein, trial status is the status of the clinical trials searchable by using keywords. Thereafter, approximately 2,092 relevant clinical trials may be identified and filtered.

Optionally, analyzing the failed clinical assets of drugs comprises eliminating any active clinical trials. Herein, the active clinical trials for a target disease recruit participants to determine safety and efficacy of drugs. Initially, those clinical trials are included which have stopped early and will not start again that is denoted by the trial status ‘Terminated’, which have stopped early before enrolling a first participant that is denoted by the trial status ‘Withdrawn’, which have stopped early but may resume again that is denoted by the trial status ‘Suspended’, and which has a status that has passed its completion date and has not been verified within the past 2 years that is denoted by the trial status ‘Unknown’. Subsequently, the first set of potential drug compositions are further determined based on included assets. The corresponding trial status for the included assets considered for analysis to procure the first set of potential drug composition is given by ‘Business decision’, ‘Lost interest’, ‘Change in practice’, ‘Resources’, ‘Change in study design’, ‘Sponsor decision’, ‘Key staff left’, ‘Insufficient data’, ‘Sponsor Decision’, and ‘Funding’. Furthermore, excluded assets are lack of efficacy of the drugs in the clinical trial, severe toxicity or adverse events and the relevancy for treatment of a disease. Subsequently, the active clinical trials on the target disease denoted by the trial status ‘Active’, the active clinical trials which are recruiting participants denoted by the trial status ‘Recruiting’, the active clinical trials not yet recruiting participants denoted by the trial status ‘Not yet recruiting’, the active clinical trials in which participants are enrolled by invitation studies denoted by the trial status ‘Enrolling by invitation studies’ and so forth are excluded and not considered further. Thereafter, after filtering and procuring the first set of potential drug composition, the first set of potential drug composition is further classified as small molecules drugs, unspecified drugs, and biologics or non-biologics. Herein, small molecule drugs are relatively simple chemical compounds and can be manufactured by chemical synthesis. Furthermore, unspecified drugs are non-specific drugs which can be tested for a range of target diseases. Additionally, biologics is a drug produced from living components of living organisms, and non-biologics are drugs produced through a fully synthetic process. Consequently, the first set of potential drug compositions that is procured is used for further evaluation and prioritization.

Continuing the example above, after filtering using trial status mentioned in the present disclosure for a target disease such as ‘Pancreatic cancer’, approximately 76 drugs may be identified. Herein, out of the 76 drugs, 52 were small molecules drugs, 6 were unspecified drugs and 17 were biologics and non-biologics.

Optionally, the discovery engine is configured to perform differential gene expression (DEG) analysis on normalized target-disease-related data acquired from omics databases. Herein, differential gene expression analysis refers to the examination and interpretation of differences among genes in abundance of gene transcripts within a transcriptome, wherein transcriptome refers to RNA transcribed from a particular genome under investigation in a given condition at a time. Notably, the omics databases are used to retrieve patient samples for data aggregation and machine learning for DEG. Additionally, genes are scored based on features. Moreover, target prioritizing of the genes is performed using algorithm-based ranking, pathway and gene function relevancy, literature mining (LM) for relevancy and druggability assessments. Herein, algorithm-based ranking includes PageRank algorithm, community ranking using Community Ranker and evidences. Herein, evidences are the number of adverse events reported of the potential drug compositions, number of Computed Tomography (CT) reports found, publications and/or other published reference. Additionally, pathway and gene function relevancy include gene function (oncology related), pathways and disease enrichment. Moreover, literature mining for relevancy includes LM validation for target regulation, LM validation for target ability for disease. Furthermore, druggability assessments include target class clustering and identification of most promising target groups. Lastly, drugs are mapped for druggable targets.

Optionally, performing differential gene expression analysis on normalized target-disease-related data acquired from omics databases comprises:

collecting target-disease-related data from omics databases;

normalizing the target-disease-related data to eliminate technical errors therefrom, wherein normalization is performed using at least one of: LOWESS Normalization, quantile normalization;

performing differential gene expression analysis on the normalized target-disease-related data;

prioritizing markers identified in differential gene expression analysis using at least one of: centrality algorithms, pathway and gene function relevancy, druggability assessments, manual scientific and prioritization.

Optionally, in this regard, the target-disease-related data is collected from omics databases, such as for example The Cancer Genome Atlas (TCGA) databases. Herein, the target-disease-related data comprises samples which are classified into datasets. Furthermore, the datasets include identification numbers of samples along with disease samples and control samples. Herein, the disease samples consist of pre-treated samples and the control samples consist of healthy samples from the target-disease-related data. Additionally, the disease samples and the control samples are included in the differential gene expression analysis.

Optionally, in this regard, the target-disease-related data is normalized to eliminate technical errors. Furthermore, the normalization is performed using either Locally Weighted Scatterplot Smoothing (LOWESS) normalization or quantile normalization. Notably, several technical errors may occur during microarray experimental procedures. Herein, the microarray experimental procedure looks for changes in gene expression across a factor of interest. Moreover, the microarray experimental procedure provides artifacts such as irregular spot printing, non-uniform intensity of fluorescent compound, dusty arrays, purification errors, difference in efficiency of labelling via fluorescent dyes, hybridization efficiencies, and systematic biases in quantified expression levels. Furthermore, these artifacts have bearings on capturing data leading to different measurements of the same expression values. Hence, it is important to eliminate these technical errors prior to any downstream analysis. Beneficially, the normalization plays an important role in reducing potential technical errors, such as for example potential systematic noises. Moreover, performance of the LOWESS normalization and the quantile normalization is assessed by revelation of systematic intensity-dependent effect in measurements taken from disease samples and control samples. Subsequently, a scatter plot using smoothScatter method is used before and after normalization to generate a smoothed color density representation of the scatter plot. Herein, a LOWESS smoothed line is employed to visually show bias. Additionally, the bias is the difference between the before and after normalization of microarray experimental procedures. Furthermore, two-channel microarrays are used in microarray experimental procedures for efficient comparison between the before and after normalization scatter plots.

Furthermore, the purpose of LOWESS normalization is to estimate the bias using a nonparametric curve known as local weighted regression. Subsequently, at each point on the scatter plot, median value is adjusted by subtracting the estimated bias at the same value of the microarray. Additionally, LOWESS normalization operates on individual chips, that is within-chip normalization. Notably, LOWESS normalization makes measures comparable across chips as well, that is between-chip normalization. Moreover, LOWESS normalization can perform locally linear fits in a robust manner which is not affected by outliers in the scatter plot of the microarray experimental procedures.

Furthermore, the quantile normalization is a simple non-parametric normalization method initially proposed for single-channel arrays. Additionally, the quantile normalization is a between-array normalization method that makes distribution of all arrays identical in statistical properties. Typically, the quantile algorithm maps every expression value on each chip to the corresponding quantile of a reference distribution that is determined by pooling across distributions of all individual chips. Moreover, a quantile-quantile plot is plotted to visualize the distribution of two data vectors. Herein, the quantile-quantile plot of the visualization of the distribution of the two data vectors is the same only if the quantile-quantile plot is a straight diagonal line. Furthermore, quantile normalization explicitly assumes that the distribution of gene expression measures is identical across the samples. Consequently, quantile normalization method has been used in the present disclosure to determine the first set of potential drug compositions.

Optionally, the differential gene expression analysis is performed on the normalized target-disease-related data. Herein, the differential gene expression analysis is performed on input data, wherein the input data comprises raw data and normalized target-disease-related data. The input data is divided into multiple samples. For instance, there may be two samples, a control sample and a case sample. Additionally, the expression values of a gene are normalized to logarithm to the base 2-fold change (log 2FC), wherein the logarithmic fold change between two conditions is calculated. Herein, logarithmic fold change is defined as a score which evaluates the average logarithmic ratio between two conditions. Subsequently, for the control sample and the case sample, differential expression is evaluated in terms of logarithm to the base 2 (log 2n), wherein both the samples are ensured to be in log 2n form. Thereafter, mean is calculated for both the control sample and the case sample, for only the control samples and for only the case samples. Herein, control mean is the mean of only the control samples, and case mean is the mean of only the case samples. Subsequently, the control mean is subtracted from the case mean in order to calculate the change in expression of the case samples with respect to the control samples. Consequently, the log 2FC of the case sample and the control sample is formulated. Herein, subtraction is equivalent to division on normal mathematical values.

Herein, the differential gene expression analysis is performed on input data, wherein the input data comprises raw data and normalized target-disease-related data. The input data is divided into multiple samples. For instance, there may be two samples, a control sample and a case sample. Additionally, the expression values of a gene are normalized to logarithmic to the base 2-fold change (log 2FC), wherein the logarithmic fold change between two conditions is calculated. Herein, logarithmic fold change is defined as a score which evaluates the average logarithmic ratio between two conditions. Subsequently, for the control sample and the case sample, differential expression is evaluated in terms of logarithm to the base 2 (log 2n), wherein both the samples are ensured to be in log 2n form. Thereafter, mean is calculated for both the control sample and the case sample, for only the control samples and for only the case samples. Herein, control mean is the mean of only the control samples, and case mean is the mean of only the case samples. Subsequently, the control mean is subtracted from the case mean in order to calculate the change in expression of the case samples with respect to the control samples. Consequently, the log 2FC of the case sample and the control sample is formulated. Herein, subtraction is equivalent to division on normal mathematical values.

Optionally, in this regard, markers are identified in differential gene expression analysis and prioritized using at least one of the centrality algorithms, pathway and gene function relevancy, druggability assessments or manual scientific and prioritization. Additionally, the first set of the potential drug compositions based on the prioritized markers are considered for further analysis. Herein, the centrality algorithms comprise identification based on p-value, betweenness centrality, PageRank algorithm, community ranking using Community ranker, and evidence. Moreover, the p-value is the probability of obtaining test results in null hypothesis significance testing, wherein the test results should be at least as extreme as the results actually observed, under assumption that the null hypothesis is correct. Furthermore, statistical testing is used to control false-positives comparison of small statistical tests of differential expression applied to microarrays. Furthermore, the betweenness centrality captures how much a given node is in-between others. Herein, the node is denoted by ‘u’. Additionally, the betweenness centrality is measured with a number of shortest paths between any couple of nodes that passes through a target node. Herein, the target node is determined by ‘σ_{v,w}(u)’. Subsequently, a score is obtained which is moderated by the total number of shortest paths existing between any couple of nodes. Herein, the total number of shortest paths existing between any couple of nodes is denoted by ‘σ_{v,w}’. The formula to determine betweenness centrality, wherein the function of between centrality is denoted by ‘B(u)’ is given by

$$B ( u ) = {\sum }_{u \neq v \neq w} \frac{{σ}_{v , w} ( u )}{{σ}_{v , w}}$$

Furthermore, the PageRank algorithm is used to determine the popularity of any molecule. Moreover, Community Ranker performs community ranking which is based on the number of molecules connected to any molecule in a network. Additionally, the molecules with more connected molecules are ranked higher. Lastly, evidence is collected from relevant literature and co-occurrence of identified marker and target-disease in abstract and title of articles.

Furthermore, in the pathway and gene relevancy, each potential marker is evaluated with the target disease based on the pathways, wherein the pathway is determined from a Pathway database. Additionally, pathways associated with target disease are given more weightage and ranking. For instance, a first marker, a second marker and a third marker have corresponding pathways. Herein, the first marker has a pathway of ‘4’, the second marker has a pathway of ‘8’ and the third marker has a pathway of ‘3’. Hence, more weightage is given to the second marker. Furthermore, while performing draggability assessment, in case the first set of potential drug compositions identified on the marker reaches a trial stage, then ‘1’ is marked. Additionally, in case the draggability assessment on the first set of potential drug compositions identified on the marker does not comprise any clinical evidence, then ‘0’ is marked. Furthermore, number of publications in Life Science domain are checked for each marker for prioritizing and the target disease. Therefore, in case the prioritized marker has a higher number of publications with respect to the target disease, the prioritized marker should have a high probability of it being relevant to the target disease.

The discovery engine is configured to evaluate effects of known drugs used for diseases similar to the target disease. Herein, an adverse events database is created which includes adverse events (AE) reported for clinical drugs from various sources. Furthermore, the various sources comprise patient forums such as for example FDA Adverse Event Reporting System (FAERS) Public Dashboard, pharmacovigilance databases such as for example VigiAccess® and VigiBase®, adverse events database such as for example SIDER and PharmGKB®, clinical trials. Herein, information is extracted from clinical trial treatment arms during clinical trials and textual information based on adverse event mapping is filtered and identified as adverse events related to the clinical trials. Furthermore, names of drugs are normalized based on the drug ontology database of Innoplexus®, names of the adverse events are normalized with reference to the Medical Dictionary for Regulatory Activities (MedDRA). Subsequently, the normalized names of the adverse events are used to prepare a proprietary ‘Adverse Event (AE) Database’ to use for further analysis in the present disclosure. Presently, approximately 2,228,248 adverse events entries for drugs are available in the AE Database. Additionally, all terminology regarding the target disease is searchable on the AE Database and is made to pass through an AE based repurposing pipeline.

Optionally, evaluating effect on known drugs is carried out using at least one of the

Fingerprint approach

Clinical trial adverse events approach

Optionally, in this regard, fingerprint approach is an Adverse Events (AE) Based fingerprint pipeline. Herein, tuples are prepared for the first set of potential drug composition and for associated adverse events. Subsequently, the tuples are ranked based on the evidences. Thereafter, a matrix of these tuples with enriched drug and adverse events features are formed. Additionally, the first set of potential drug compositions with similar adverse events are mapped together and ranked with Jaccard index. Herein, Jaccard index is used to gauge the similarity and diversity of tuples within the matrix. Moreover, the first set of potential drugs are further clustered based on investigational and clinical therapeutic areas and drug-indication pairs are identified.

Optionally, in this regard, the clinical trial adverse events approach is used to identify common adverse events reported with the first set of potential drug composition and number of adverse events reported with placebo in the clinical trials. In case the fold change is greater than 2 and the number of adverse events matched or the number of reports or patient matched is greater than 5, then the first set of potential drug compositions is considered to be the preliminary drug with a new indication list. Herein, the indication list and the drug-indication pairs are cross-checked with the CT reports and the publications.

Furthermore, if the indication list and the drug-indication pairs are matched with only the CT reports, fingerprinting is generated. However, if the indication list and the drug-indication pairs are not matched with the publications, a high weightage is given to the drug-indication pairs and are scored based on biological evidence. Herein, the biological evidences comprise Molecular Docking, omics score, analysis of patents, score based on evidence from literature, and genome-wide association study (GWAS) scoring. Herein, Molecular Docking comprises information regarding relevant protein and the first set of potential drug compositions interactions, the omics score maps expression present in the datasets. Furthermore, the patent that is analyzed should be novel and confirm that no patent exists against the identified first set of potential drug compositions and the indication list. Additionally, scores based on evidence from literature are performed based on the number of preclinical evidences. Moreover, GWAS scoring suggests the role of a number of mutations in the new indication list. Therefore, a list of alternative indications for the first set of potential drug compositions based on similar adverse events is procured.

The discovery engine is configured to perform a network-based analysis to identify repurposable drugs based on similar targets and indirect pathways for the target disease. Herein, the network-based analysis is performed to provide insights regarding repositioning of drugs in context of the target disease. Furthermore, network-based analysis improves the knowledge regarding multiple actions of drugs. Additionally, network-based analysis improves suggestion and identification of repurposable drugs. Herein, repurposable drugs are existing drugs which are investigated further to determine new disease-related purposes. Furthermore, repurposable drugs are a strategy to treat diseases which are neglected due to reduced number of required clinical trials and/or due to the suddenness of the onset of the disease among the masses.

Optionally, the network-based analysis is performed on a multi-entity network comprising nodes representing drugs, targets, diseases and pathways. Herein, the network-based analysis is performed using a network of drugs, targets, diseases and pathways. Additionally, only the drugs indirectly associated with the target disease are identified to be repurposable drugs and are used for further analysis. Continuing with the above example, approximately 2 pathways, 400 proteins and 399 drugs were identified for the target disease “Pancreatic cancer” which are directly or indirectly related with pancreatic cancer. Consequently, approximately 134 indirectly associated drugs with pancreatic cancer were considered for further analysis for the most suitable repurposable drug against pancreatic cancer.

The method comprises asset prioritization to filter the first set of potential drug compositions to determine at least one potential drug composition for the target disease. Herein, asset prioritization is used to find a new suggestion for the first set of potential drug compositions, suggest a combination of one or more potential drug compositions to treat the target disease, assess risk profile of the first set of potential drug compositions to compare with other development options and so forth.

Optionally, asset prioritization is performed to filter the first set of potential drug compositions to determine at least one potential drug composition comprises filtering based on

potential drug compositions with no active clinical trials against the target disease;

inhibitory mechanisms of the potential drug compositions;

experimental support for the effectiveness against the target disease;

adverse events reported in public domain against the potential drug composition;

overall survival reports related to the target disease; and

binding affinity of the potential drug compositions.

Optionally, in this regard, potential drug compositions with no active clinical trials against the target disease are filtered. Furthermore, the potential drug compositions are not listed in the drug pipeline of an individual pharmaceutical company or the entire pharmaceutical industry. Additionally, inhibitory mechanisms of the potential drug compositions are filtered. Herein, target insights are acquired from the datasets of the omics databases. Moreover, evidence is also collected in support of the potential drug compositions. Subsequently, adverse events reported in public domain against the potential drug composition are filtered, wherein no serious adverse events or less serious adverse events reported in public domain and scientific evidence of the adverse event is accumulated. Furthermore, overall survival reports comprising information regarding survival benefits for the target disease and the increase in survival rates or progression free survival is determined. Additionally, filtering is performed based on binding affinity. Herein, binding affinity is the strength of binding interaction between a single biomolecule to a drug. Moreover, the binding affinity may be determined by determining bioassays, mutation, structural insights and half maximal inhibitory concentration (IC₅₀) of the potential drug composition. Herein, bioassay is used to measure the biological activity and effects of the potential drug composition. Furthermore, mutation of a potential drug composition may turn the potential drug composition from an antagonist to an agonist. Additionally, structural insights determine the structural basis of the potential drug composition. In addition, IC₅₀ provides a measure of potency of the potential drug composition in inhibiting a specific biological or biochemical function.

The method comprises validating at least one potential drug composition for the target disease based on biological evidence and differential expression analysis. Herein, at least one potential drug may be identified to be a repurposable drug based on scientific validation of the first set of potential drug compositions using network-based analysis. Herein, a biological network is created based on the biological evidence to associate the at least potential drug composition with the target disease, which is further explored and probable associations are determined based on the biological evidence-based relationships. Furthermore, at least one potential drug composition is determined using the AE based repurposing pipeline and datasets from omics databases.

Optionally, the potential drug composition is associated with a plurality of targets using the biological evidence and the differential expression analysis to validate at least one potential drug composition. Herein, the discovery engine identifies and clusters various known and potential associations based on filters. Furthermore, the filters comprise scores given to the plurality of targets and druggability which may be leveraged for most significant associations of the potential drug composition with the plurality of targets. Herein, the scores are given to the plurality of targets using statistical scoring approach. Furthermore, druggability is used in the identification of the potential drug composition to describe a biological target that is known to or is predicted to bind with high affinity to a drug.

Optionally, one or more potential drug compositions is evaluated which is to be used in combination with each other at a specific ratio. Herein, the target disease is provided as an input to Gene Ontology (GO), wherein Gene Ontology comprises the relationship between biological domain to molecular function, cellular component and biological process. Subsequently, the GO yields a gene set of the target disease.

Furthermore, a Disease Ontology (DO) semantically integrates diseases and medical vocabularies into a single structure for classification of diseases, and provides semantic information related to the target disease as input to the GO. Additionally, similarities are determined between the gene set of the target disease and semantic information related to the target disease and publishes the similarities in a publication. Moreover, the publication further comprises the potential drug compositions. Consequently, the publications publish the potential drug composition based on biological evidence, experimental model, dosage of the potential drug composition, combination of the potential drug composition with the one or more potential drug composition and the specific ratio of the one or more potential drug compositions with each other.

In another embodiment, in order to identify one or more potential drug composition for a disease, the present invention employs a relational database, wherein the processor identifies a plurality of associated targets from the relational database. After identification of the plurality of associated targets, the processor identifies relevant targets for the disease based on the criteria including at least one of, but not limited to literature, network, clinical trial, expression, variant/mutation, target class/family, drug Class, experimental studies, binding affinity, sequence/structure-based similarity, targetability, antigenicity, drug resistance, and BBB penetration.

Optionally, the targets are scored using TF-IDF based on co-occurrence in one or more literature. In information retrieval, TF-IDF (frequency-inverse document frequency) is a numerical statistic that is intended to reflect how important a word is to a document in a collection or corpus. Optionally, the processor is configured to employ TF-IDF as a weighting factor in searches of information retrieval, and text mining.

Optionally, the processor identifies the relevant targets from the associated targets based on (i) Network formation, (ii) Betweenness centrality. In the network formation, the processor forms a network using Disease targets and interactions therewith in human proteome. In the betweenness centrality, the processor calculates betweenness centrality to get the targets which are very well connected in the disease network and directly or indirectly impact the greatest number of targets in the disease network.

Optionally, the processor identifies the relevant targets from the associated targets based on clinical trial as the targets which have shown proven efficacy in disease are identified thereby. In an instance, the processor maps disease drugs in clinical trials and map them with the primary targets therewith. Furthermore, said targets are then scored based on the clinical trial and also whether it has been used in combination therapy or mono therapy.

CT_{drug}=Σ(count ct*phase CT)

Optionally, the processor identifies the relevant targets from the associated targets based on differential expression of gene/target in disease condition as parameter in order to determine the normal versus disease condition. More optionally, the processor identifies the relevant targets from the associated targets based on variant/mutation. In accordance with the present disclosure, a gene mutation is a change in one or more genes. Optionally, the processor identifies the relevant targets from the associated targets based on:
Pre-clinical studies: Experiments conducted on cell line (in-vitro), animal model organisms (in-vivo) and computationally (in-silico) are considered under preclinical studies.
In-vitro studies: These are the preliminary studies which includes all the cellular experiments such as cytotoxicity, in-vitro drug loading and release profile, co-localization, and so forth.
In-vivo studies: These are the experiments conducted on model organisms (like—Mouse/Mice/Rat, Monkey, Pig, etc.) in order to understand the knockdown, dose, toxicity, bioavailability, etc., before reaching the clinical trials.
In-silico studies: Computational studies such as docking, modeling, structure activity relationship, network based, machine learning, mathematical modeling, and so forth, leads the processor to predict drug combinations.

Optionally, the processor identifies the relevant targets from the associated targets based on binding affinity/energy. It will be appreciated that Drug-target residual interaction can be scored as a docking score based on binding affinity. The best fit model follows the energy minimization rule and can be calculated as binding affinity/energy.

Optionally, the processor identifies the relevant targets from the associated targets based on sequence/structure-based similarity. The sequence or structural similarity are based on known sequence or structure of the protein or drug. Optionally, the sequence similarity employs primary structure as in FASTA, BLAST or PDB sequence as received input.

In an embodiment, the processor assigns confidence score to the targets based on the above criteria to get disease relevance of the targets. Based on our confidence score, the processor identifies the point after which there is a sudden dip in the confidence score of the targets. It is further appreciated that if no such point is found all the targets are taken to the next step for further processing.

Optionally, the processor further enriches list of targets that have been assigned the confidence score. In an embodiment, the processor makes the ranked disease targets to undergo pathway enrichment, biological process (BP) enrichment, molecular functions (MF) enrichment using hypergeometric distribution. For pathway enrichment, the processor employs the inhouse database, for both BP and MF enrichment and gene ontology database. The processor is also configured to make the ranked disease targets to undergo adverse event enrichment. Herein, the two data sets are formed: a data set of drugs and their primary target and a data set of drug and their associated adverse event. Using these two data sets using overlapping drugs, adverse events are enriched for each target using statistical tests. For each target, the disclosed system takes those adverse events which have an adjusted p value cut off of<=0.05. Optionally, for each of these targets a safety score is calculated using lethality of adverse event, shared adverse event with the input target.

Target_{AE}=Σ((−log₁₀P)*lethality)

In another embodiment, the processor clusters the one or more disease targets using community clustering method (Girvan-Newman algorithm). Generally, the disease targets include two sets, cluster 1 having same group genes and cluster 2 having different group genes. In order to select the combinatorial genes which, target similar pathways, the processor selects genes from the cluster 1 and take it to the next step. In order to select combinatorial genes which target dissimilar pathways, the processor selects genes from the cluster 2 and take it to the next step (as described later in the description).

In another embodiment, after the clustering operation, the processor constructs a target matrix. Optionally, the target matrix is constructed using all the targets from the above-mentioned processing steps. The targets are mapped with the plurality of parameters such as pathway, biological process, molecular function, dysregulated gene, clinical SoC and so forth. If a target is associated with the parameters or having any synergistic interaction, the processor marks such target as 1, else the processor marks said parameter as 0. Optionally, when a target is also a primary protein of a SoC Drug, it is marked one else zero. Using the above matrix, the processor calculates the Manhattan distance between input target and other targets using the plurality of parameters.

D_{Manhattan}=|X₂−X₁|+|Y₂−Y₁|

In another embodiment, the processor is configured to make a scoring sheet which contains disease target confidence score, adverse event score, matrix score, SoC, co-expression, co-localization, and protein structure similarity.

Disease target confidence score: Herein, the processor selects top synergistic targets which have association sore in disease.
Adverse event score: Herein, the processor selects synergistic target which have low adverse event score and do not share adverse event with the input target.
Matrix score: The processor selects the targets which have the highest number of scores in the matrix, indicating that the drug targets follow association with the most plurality of parameters.
SoC: The selected targets by the processor is also a SOC gene.
Co-expression: Herein, co-expression refers to the expression of two or more than two genes together.
Protein structure similarity: Herein, protein structure similarity refer to similarity score between two proteins based on the protein structure.

Using the above-mentioned scores, the processor determines a scored list of most synergistic targets with the input target.

The present disclosure also relates to the method as described above. Various embodiments and variants disclosed above apply mutatis mutandis to the system.

It will be appreciated that the pharmaceutical composition is identified using the method and system described above. Examples of the pharmaceutical composition containing DFX include liquid (solutions, suspensions or emulsions), solid (tables, powder or capsules) with suitable composition for oral administration, and they may contain the pure compound or in combination with any carrier or other pharmacologically active compounds.

In silico studies and models for treatment of pancreatic cancer using deferasirox (DFX) shows that DFX works in treating iron toxicity by binding trivalent (ferric) iron (for which it has a strong affinity), forming a stable complex which is eliminated via the kidneys. Therefore, deferasirox or a pharmaceutical composition thereof, will be effective in the treatment of pancreatic cancer.

Optionally, the one or more chemotherapeutic agents effective against pancreatic cancer are at least one of: Albumin-bound paclitaxel (Abraxane), Capecitabine (Xeloda), Carboplatin (Paraplatin), Cisplatin, Cyclophosphamide (Cytoxan), Daunorubicin, Docetaxel, Doxorubicin, Epirubicin, Eribulin (Halaven), Gemcitabine (Gemzar), Irinotecan (Camptosar), Ixabepilone (Ixempra), Methotrexate, Mitomycin (chemical name: mutamycin), Mitoxantrone, Paclitaxel, Thiotepa, Vincristine and Vinorelbine (Navelbine).

Optionally, the ratio of DFX to chemotherapeutic agent may vary from 1:0.025 to 1:5. Optionally, deferasirox (DFX) is used to inhibit CYP3A4, UGT1A1 and UGT1A9 activity. Pancreatic cancer associated target proteins are CYP3A4, UGT1A1, UGT1A3, UGT1A9, CYP2C8 and CYP1A2. High variation in expression of CYP3A4, CYP1A1, UGT1A9 and CYP2C8 in most samples of PaCa. Deferasirox can inhibit the CYP3A4, UGT1A1 and UGT1A9 activity and increase the survival probability of pancreatic cancer patients.

Optionally, deferasirox (DFX) is employed in combination with gemcitabine (GEM) for the suppression of Ribonucleotide reductase (RR) activity.

Optionally, the treatment comprises administration of initial DFX dose of 20 mg/kg body weight of the subject.

Optionally, the treatment further comprises to increase the dose gradually, such as 90 mg, 125 mg, 180 mg, 250 mg, 360 mg and/or 500 mg per kg body weight of the subject under administration.

Administration of DFX or compositions as described herein is based on a Dosing Protocol preferably by oral delivery. Preferably, the initial delivery dose of DFX is 20 mg/kg body weight of the subject under administration Subsequently, the doses may be increased gradually, such as 90 mg, 125 mg, 180 mg, 250 mg, 360 mg and/or 500 mg (per kg body weight of the subject under administration) with a time to a maximum plasma contention (Tmax) ranging between 90 minutes to 240 minutes, a bioavailability of about 70% and volume of distribution of 14.37±2.69 litter. Short delivery times which allow treatment to be carried out without a stay in hospital are especially desirable.

Depending on the type of tumor and the developmental stage of the disease, the uses of the formulations disclosed are useful in preventing the risk of developing tumors, in promoting tumor regression, in stopping tumor growth and/or in preventing metastasis.

The correct dosage of the compound will vary according to the particular formulation, the mode of application, and the particular situs, host and tumor being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

Additionally, the therapeutic agent may include but not limited to agents or drugs used in chemotherapy, targeted therapy and/or immunotherapy.

Optionally, the treatment further comprises a time to a maximum plasma contention (Tmax) ranging between 90 minutes to 240 minutes and a bioavailability of about 70% and volume of distribution of 14.37±2.69 litre. Optionally, the treatment comprises oral administration of the composition.

### DETAILED DESCRIPTION OF THE DRAWINGS

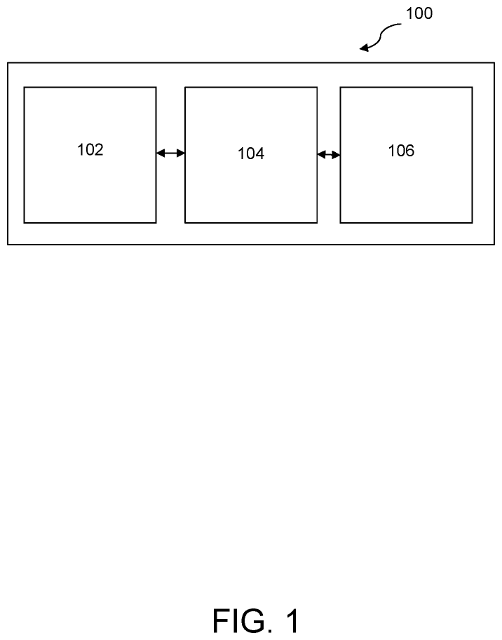

Referring to FIG. 1, there is shown a schematic illustration of a block diagram of a system 100 of determining potential drug compositions in accordance with an embodiment of the present disclosure. The system 100 comprises a database arrangement 102 and a processor 106 communicably coupled via a data communication network 104 to the database arrangement 104.

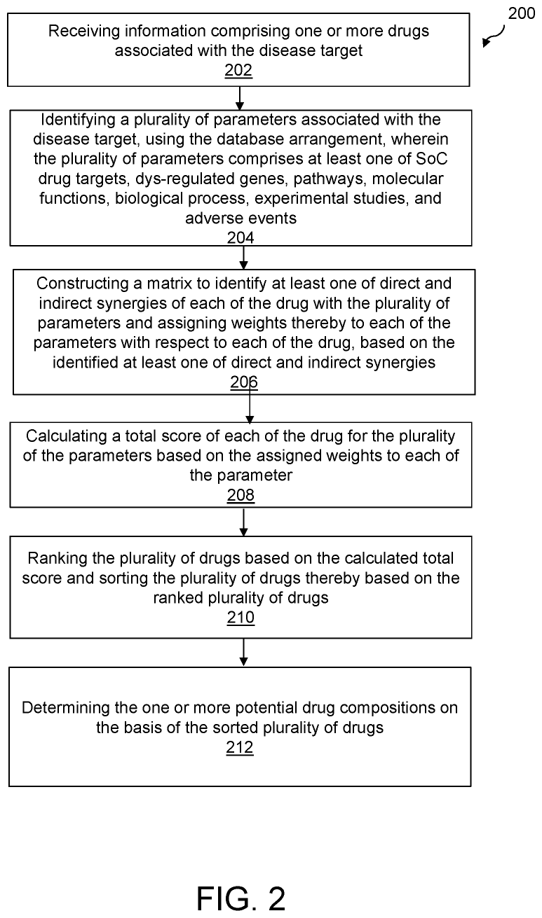

Referring to FIG. 2, there is shown a schematic illustration of the steps of method 200, in accordance with an embodiment of the present disclosure. At step 202, the method comprises receiving information comprising one or more drugs associated with the disease target. At step 204, the method comprises identifying a plurality of parameters associated with the disease target, using the database arrangement, wherein the plurality of parameters comprises at least one of SoC drug targets, dys-regulated genes, pathways, molecular functions, biological process, experimental studies and adverse events. The method further comprises constructing a matrix to identify at least one of direct and indirect synergies of each of the drug with the plurality of parameters and assigning weights thereby to each of the parameters with respect to each of the drug, based on the identified at least one of direct and indirect synergies, step 206 of the method 200. At step 208, the method comprises calculating a total score of each of the drug for the plurality of the parameters based on the assigned weights to each of the parameter. At step 210, the method comprises ranking the plurality of drugs based on the calculated total score and sorting the plurality of drugs thereby based on the ranked plurality of drugs, and at method step 212, the method comprises determining the one or more potential drug compositions on the basis of the sorted plurality of drugs.

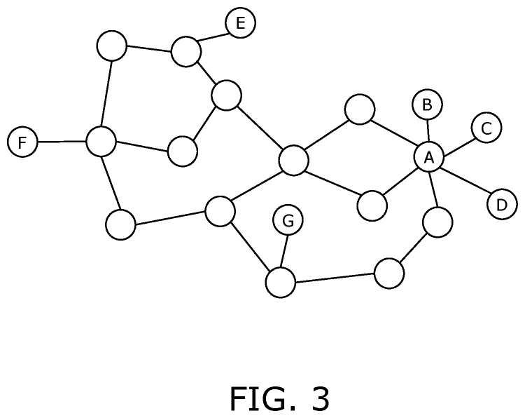

Referring to FIG. 3, illustrated is an exemplary diagram showing betweenness centrality for marker prioritization, in accordance with an embodiment of the present disclosure. The target node has a high centrality if it appears in many shortest paths. The node A shows high betweenness centrality while B, C, D, E, F and G has lowest betweenness centrality.

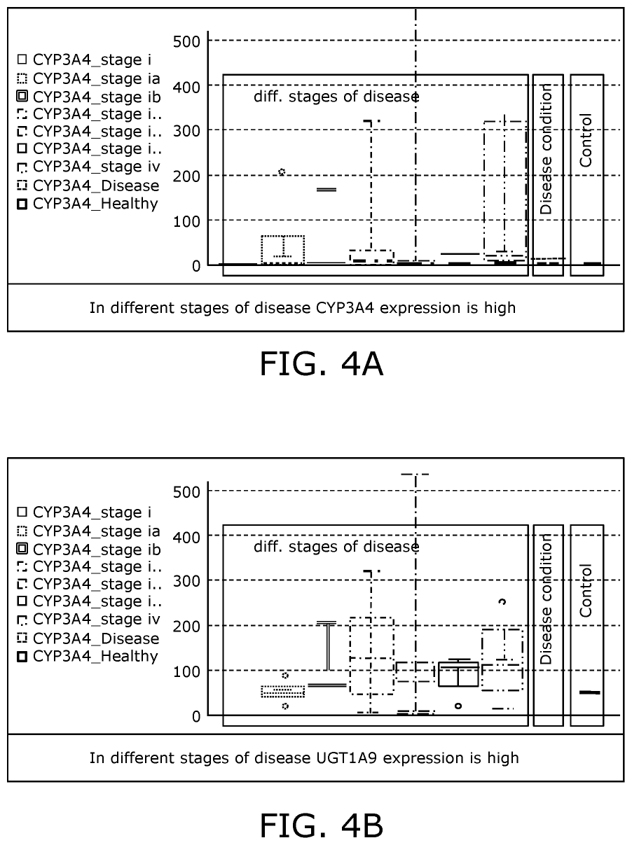

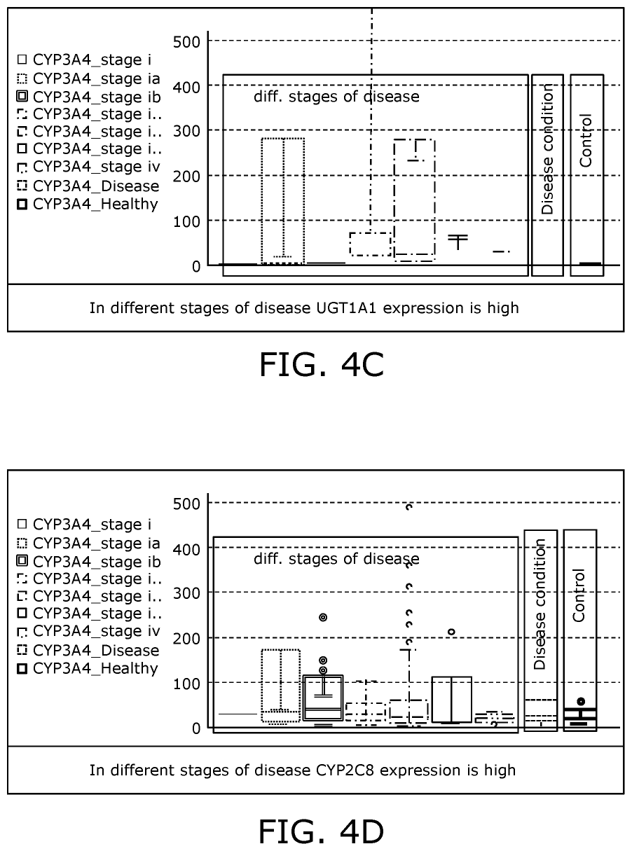

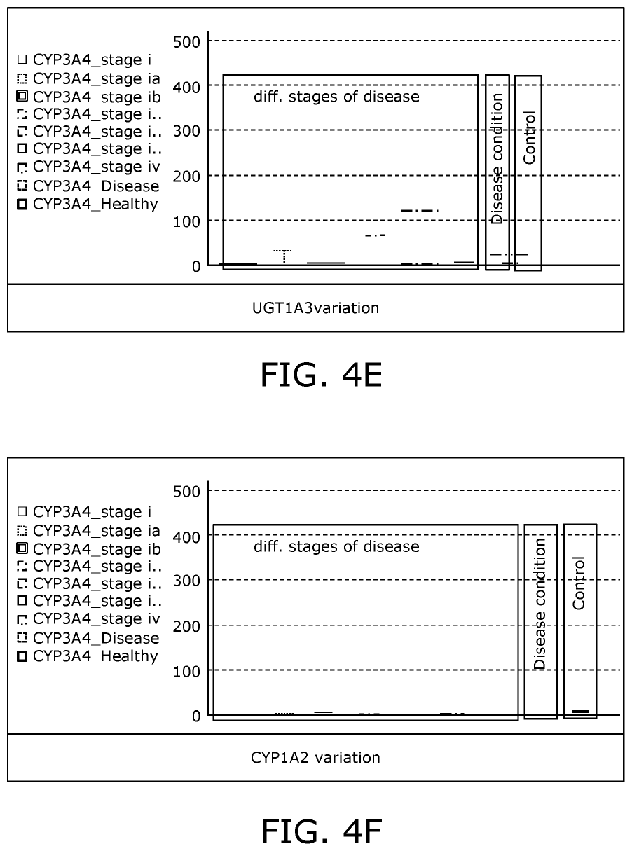

Referring to FIG. 4A-4F, illustrated are exemplary graphs showing differential expressions of DFX with pancreatic cancer associated target proteins CYP3A4, UGT1A1, UGT1A3, UGT1A9, CYP2C8 and CYP1A2, in accordance with an embodiment of the present disclosure. In the target proteins, high variation in expression is found in most sample of pancreatic cancer. The expression variation is mainly observed in stage ii, iii, and iv of the disease. Subsequently, it is identified that the expression of UGT1A1 in pancreatic cancer is high and it is directly connected to it.

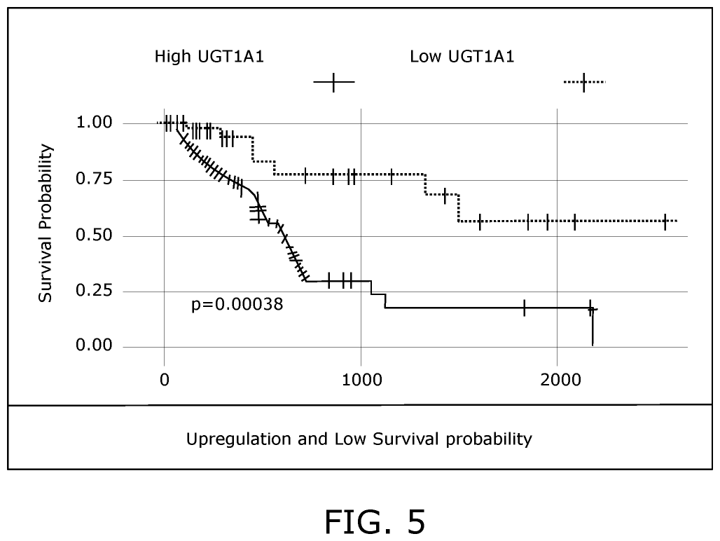

Referring to FIG. 5, illustrated is a graph showing survival probability when compared with high and low expressions of target protein UGT1A1, in accordance with an embodiment of the present disclosure. Subsequently, it is found that low expression of the target protein UGT1A1 leads to better survival probability.

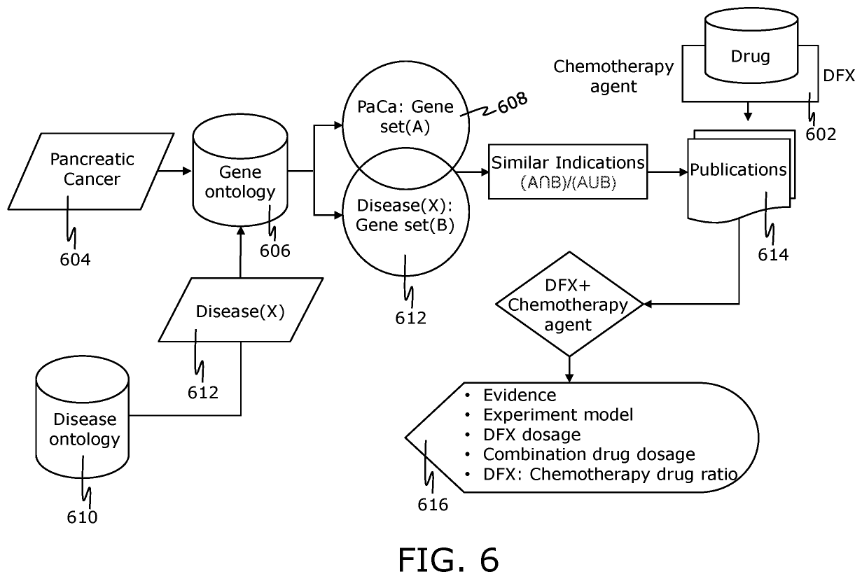

Referring to FIG. 6, illustrated is a block diagram showing evaluation of potential drug compositions 602 DFX and a chemotherapy agent for pancreatic cancer 604, in accordance with an embodiment of the present disclosure. Herein, pancreatic cancer 604 is provided as an input to Gene Ontology 606. Subsequently, the Gene Ontology 606 yields gene set 608 of pancreatic cancer 604. Moreover, a Disease Ontology 610 semantically integrates diseases and medical vocabularies into a single structure for classification of diseases, and provides semantic information 612 related to pancreatic cancer 604 as input to the Gene Ontology 606. The similarities are determined between the gene set 608 of pancreatic cancer 604 and semantic information 612 related to pancreatic cancer 604 and publishes the similarities in a publication 614. The publication 614 further comprises the potential drug compositions 602 DFX and a chemotherapy agent. Consequently, the publications 614 publish DFX with chemotherapy agent composition 602 based on certain factors 616. The factors 616 are biological evidence, experimental model, DFX dosage of the potential drug composition 614, combination drug dosage and the specific ratio.

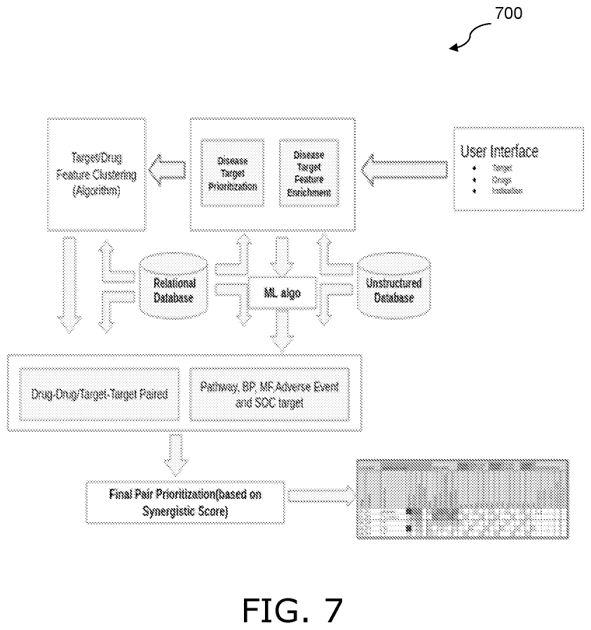

Referring to FIG. 7, illustrated is a block diagram 700 showing efficient synergistic target identification, in accordance with an embodiment of the present disclosure. In accordance with the present disclosure, the input received from the user interface may comprise target, drug, and/or indication (disease). The processor determines the plurality of targets from the relational database based on the input received from the user interface. In an embodiment, the processor is configured to assign weights to the plurality of targets based on the plurality of parameters such as literature, biological process, pathway, molecular function and so forth, as described in FIG. 7. Thereafter, the processor further clusters the plurality of targets using community clustering algorithm method. Through the community clustering method, the processor generates a plurality of clusters of the disease targets. Optionally, the processor analyzes the generated plurality of clusters, wherein each of the cluster is analyzed to identify synergies of each of the cluster with the other cluster from the plurality of clusters. Specifically, the processor evaluates parameters such as adverse events, pathway, molecular functions and so forth of a drug associated with a particular target in a cluster, and said parameters are analyzed with the targets of other clusters from the plurality of clusters. Based on the analysis of the plurality of clusters, the processor generates synergistic scoring of each of the target associated with each of the cluster. Furthermore, the matrix is generated by the processor, wherein the matrix maps the targets and/or the associated drugs with each of the parameter from the plurality of parameters and based on the mapping of the drug and/or targets with each of the plurality of parameters, the one or more potential synergistic target combination is determined. Optionally, the one or more associated potential drug combination is also determined based on the matrix and clustering process.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system implemented in a computing device for generating potential drug compositions for a disease target, the system comprises:
a database arrangement; and
a processor communicably coupled via a data communication network to the database arrangement, wherein the processor is configured to:
receive information comprising one or more drugs associated with the disease target;
identify a plurality of parameters associated with the disease target, using the database arrangement, wherein the plurality of parameters comprises at least one of SoC drug targets, dys-regulated genes, pathways, molecular functions, biological process, experimental studies and adverse events;
construct a matrix to identify at least one of direct and indirect synergies of each of the drug with the plurality of parameters and assign weights thereby to each of the parameters with respect to each of the drug, based on the identified at least one of direct and indirect synergies;
calculate a total score of each of the drug for the plurality of the parameters based on the assigned weights to each of the parameter;
rank the plurality of drugs based on the calculated total score and sort the plurality of drugs thereby based on the ranked plurality of drugs; and
determine the one or more potential drug compositions on the basis of the sorted plurality of drugs.

2. The system of claim 1, wherein the processor is further configured to validate the at least one potential drug composition for the target disease based on biological evidence and differential expression analysis.

3. The system of claim 1, wherein the received information also comprises the drug targets associated therewith each of the drug.

4. The system of claim 1, wherein each of the parameter from the plurality of parameters comprises a library of associated parameters.

5. The system of claim 4, wherein the processor is configured to identify at least one of the direct and indirect synergies of each of the drug with each of the parameter from the library of associated parameters.

6. The system of claim 1, wherein the processor is configured to identify at least one of the direct and indirect synergies of each of the drug with each of the parameter based on one or more ontologies.

7. The system of claim 6, wherein the one or more ontologies corresponds to at least a drug ontology, a protein ontology and a gene ontology.

8. The system of claim 1, wherein the processor is further configured to employ asset prioritization to filter the first set of potential drug compositions to determine at least one potential drug composition for the target disease.

9. The system of claim 1, wherein the asset prioritization to filter the one or more potential drug compositions to determine at least one potential drug composition comprises filtering based on:
potential drug compositions with no active clinical trials against the target disease;
inhibitory mechanisms of the potential drug compositions;
experimental support for the effectiveness against the target disease;
adverse events reported in public domain against the potential drug composition;
overall survival reports related to the target disease; and
binding affinity of the potential drug compositions.

10. The system of claim 1, wherein the processor is further configured to associate the one or more potential drug composition with a plurality of targets using the biological evidence and the differential expression analysis to validate the at least one potential drug composition.

11. The system of claim 1, wherein the processor is configured to evaluate one or more potential drug compositions to be used in combination with each other at a specific ratio.

12. A method for generating potential drug compositions for a disease target, the method comprises:
receiving information comprising one or more drugs associated with the disease target;
identifying a plurality of parameters associated with the disease target, using the database arrangement, wherein the plurality of parameters comprises at least one of SoC drug targets, dys-regulated genes, pathways, molecular functions, biological process, experimental studies and adverse events;
constructing a matrix to identify at least one of direct and indirect synergies of each of the drug with the plurality of parameters and assigning weights thereby to each of the parameters with respect to each of the drug, based on the identified at least one of direct and indirect synergies;
calculating a total score of each of the drug for the plurality of the parameters based on the assigned weights to each of the parameter;
ranking the plurality of drugs based on the calculated total score and sorting the plurality of drugs thereby based on the ranked plurality of drugs; and
determining the one or more potential drug compositions on the basis of the sorted plurality of drugs.

13. The method of claim 12, wherein the method comprises validating the at least one potential drug composition for the target disease based on biological evidence and differential expression analysis.

14. The method of claim 12, wherein the received information also comprises the drug targets associated therewith each of the drug.

15. The method of claim 12, wherein each of the parameter from the plurality of parameters comprises a library of associated parameters.

16. The method of claim 15, wherein the method comprises identifying at least one of the direct and indirect synergies of each of the drug with each of the parameter from the library of associated parameters.

17. The method of claim 12, wherein the method comprises identifying at least one of the direct and indirect synergies of each of the drug with each of the parameter based on one or more ontologies, and wherein the one or more ontologies corresponds to at least a drug ontology, a protein ontology and a gene ontology.

18. The method of claim 12, wherein the method comprises employing asset prioritization to filter the first set of potential drug compositions to determine at least one potential drug composition for the target disease.

19. The method of claim 12, wherein the asset prioritization to filter the one or more potential drug compositions to determine at least one potential drug composition comprises filtering based on:
potential drug compositions with no active clinical trials against the target disease;
inhibitory mechanisms of the potential drug compositions;
experimental support for the effectiveness against the target disease;
adverse events reported in public domain against the potential drug composition;
overall survival reports related to the target disease; and
binding affinity of the potential drug compositions.

20. The method of claim 12, wherein the method comprises evaluating one or more potential drug compositions to be used in combination with each other at a specific ratio.
